(19) United States
(12) Patent Application Publication
(10) Pub. No.: US 2005/0244340 A1
(43) Pub. Date: Nov. 3, 2005

(54) Dry powder compositions

(76) Inventors: Pallav Arvind Bulsara, Research Triangle Park, NC (US); Trevor Charles Roche, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/511,042

(22) PCT Filed: Apr. 10, 2003
(86) PCT No.: PCT/GB03/01542

§ 371 Date: May 31, 2005

(30) Foreign Application Priority Data
Apr. 13, 2002 (GB) 0208608.0

(51) Int. Cl. A61L 9/04; A61K 9/14; A61K 31/7024

(52) U.S. Cl. 424/46; 514/54; 514/53

(57) ABSTRACT

Dry powder pharmaceutical compositions having improved storage stability, dry powder inhalers comprising the same and their use in the treatment of respiratory disorders by inhalation.

This invention relates to dry powder pharmaceutical compositions, and their use in the treatment of respiratory disorders by inhalation. The invention also relates to dry powder inhalers comprising the same. More particularly, this invention relates to dry powder pharmaceutical compositions having improved stability.

Dry powder inhalers (DPI's) are well known devices for administering pharmaceutically active agents to the respiratory tract. Consequently, they are particularly suitable when used for the administration of active agents in the treatment of diseases such as asthma, bronchitis, chronic obstructive pulmonary disease (COPD), emphysema, rhinitis etc. Since the drug acts directly on the target organ much smaller quantities of the active ingredient may be used, thereby minimising any potential side effects.

Dry powder compositions for use as inhalable medicaments in DPI's typically comprise a pharmaceutically active agent intimately admixed with an excess of pharmaceutically acceptable excipient or excipients (often called carrier(s)). Such excipients serve not only to dilute the quantity of active agent administered in each dose but also to establish acceptable manufacture of the powder mixture and aid in the aerosolisation of the drug. Such a high proportion of excipient will essentially determine the properties of the powder formulation, particularly the manufacturing characteristics.

A problem associated with the use of dry powder pharmaceutical compositions of this type is that they can be susceptible to poor stability performance due to moisture ingress. For example, significant deterioration in the fine particle dose (FPD), namely that which has the potential to penetrate into the lower airways of the lung, is often observed upon protracted exposure of such compositions to conditions of elevated temperature and humidity.

Patent application WO 00/28979 (SkyePharma) describes one approach to overcome the above noted problems. It is claimed that dry powder formulations comprising a pharmaceutically active agent, an inhaled vehicle of non-inhalable particle size and magnesium stearate have improved storage stability under extreme (temperature and humidity) conditions.

We have now discovered that dry powder pharmaceutical compositions containing certain derivatised carbohydrates demonstrate surprisingly enhanced stability performance. Such compositions therefore represent an alternative solution to the above noted problem.

The present invention therefore provides, in a first aspect, the use of particulate derivatised carbohydrates in dry powder pharmaceutical compositions for inhalation therapy in order to improve stability performance.

The present invention also provides for the use of particulate derivatised carbohydrates in dry powder pharmaceutical compositions for inhalation therapy in order to eliminate or reduce the detrimental effect on fine particle dose caused by storage of said compositions.

The particulate derivitised carbohydrates can be in amorphous or crystalline particulate form. Preferably the particulate derivitised carbohydrate is in crystalline form.

Dry powder pharmaceutical compositions for inhalation therapy comprising particulate derivatised carbohydrates are believed to be novel. Consequently, the present invention further provides for a dry powder pharmaceutical composition suitable for inhalation therapy, with improved storage stability performance, comprising a pharmaceutically active agent, an excipient and a derivatised carbohydrate in particulate form. Suitably the derivitised carbohydrate is in crystalline form.

It is to be understood that the dry powder pharmaceutical compositions according to this invention include not only those in which the components are incorporated as individual particles but also those including matrix particles of more than one component. For example, matrix particles of pharmaceutically active agent and a derivatised carbohydrate or matrix particles of excipient and a derivitised carbohydrate may be utilised. Such matrix particles can be prepared by solid dispersion technology e.g. co-precipitation and particle coating methods which are familiar to those skilled in the art. Suitably, the components are incorporated as individual particles.

The term “derivatised carbohydrates” is used herein to describe a class of molecules in which at least one hydroxyl group of the carbohydrate group is substituted with a hydrophobic moiety via either ester or ethers linkages. All isomers (both pure and mixtures thereof) are included within the scope of this term. Mixtures of chemically distinct derivatised carbohydrates may also be utilised.

Suitably, the hydroxyl groups of the carbohydrate may be substituted by a straight or branched hydrocarbon chain comprising up to 20 carbon atoms, more typically up to 6 carbon atoms. The derivatised carbohydrates can be formed by derivitisation of monosaccharides (e.g. mannitol, fructose and glucose) or of disaccharides (e.g. maltose, trehalose, cellobiose, lactose and sucrose). Derivatised carbohydrates are either commercially available or can be prepared according to procedures readily apparent to those skilled in the art.

Non limiting examples of derivatised carbohydrates include cellobiose octaacetate, sucrose octaacetate, lactose octaacetate, glucose pentaacetate, mannitol hexaacetate and trehalose octaacetate. Further suitable examples include those specifically disclosed in patent application WO 99/33853 (Quadrant Holdings), particularly trehalose diisobutyrate hexaacetate. A particularly preferred derivatised carbohydrate is cellobiose octaacetate, most preferably α-D cellobiose octaacetate.

Typically, the aerodynamic size of the derivatised carbohydrates will be between 0.1 and 50 μm, and more particularly 1-20 μm. The derivatised carbohydrates for use in the preparation of compositions in accordance with this invention are typically micronised but controlled precipitation, supercritical fluid methodology and spray drying techniques familiar to those skilled in the art may also be utilised.

The derivitised carbohydrate may be present in a concentration of 0.01-99% by weight of the total composition. Suitably the derivatised carbohydrate is present in a concentration of 0.01-50% by weight of the total composition, preferably 1-20%.

The pharmaceutically active agent can be any therapeutic molecule in dry powder form that is suitable to be administered by inhalation. In the field of inhalation therapy, the term “suitable to be administered by inhalation” is generally taken to mean therapeutic molecules having an aerodynamic diameter between 0.1 and 10 μm, and more particularly 1-5 μm. Particles of the desired particle size for inhalation are conventionally prepared by micronisation. Other methods of producing such particles are also known in the art. Therefore, such particles can also be prepared using controlled precipitation methods (e.g. methods described in patent applications WO 00/38811 and WO 01/32125 (Glaxo Group Limited)), using supercritical fluid methodology or by spray drying techniques. The present invention provides no limitation on the method by which the therapeutic molecule is made suitable to be administered by inhalation.

Examples of pharmaceutical active agents suitable for inhalation therapy include analgesics, e.g., codeine, dihydromorphine, ergotamine, fentanyl or morphine; anginal preparations, e.g., diltiazem; anti-allergics, e.g., cromoglycate (e.g. as the sodium salt), ketotifen or nedocromil (e.g. as the sodium salt); anti-infectives e.g., cephalosporins, penicillins, streptomycin, sulphonamides, tetracyclines and pentamidine; anti-histamines, e.g., methapyrilene or loratadine; anti-inflammatories, e.g., beclomethasone (e.g. as the dipropionate ester), fluticasone (e.g. as the propionate ester), flunisolide, budesonide, rofleponide, mometasone (e.g. as the furoate ester), ciclesonide, triamcinolone (e.g. as the acetonide), 6α,9α-difluoro-11 β-hydroxy-16-methyl-3-oxo-17α-propionyloxy-androsta-1,4-diene-17β-carbothioic acid S-(2-oxo-tetrahydro-furan-3-yl) ester (also named as 6α,9α-Difluoro-17α-[(2-furanylcarbonyl)oxy]-11β-hydroxy-16-methyl-3-oxo-androsta-1,4-diene-17β-carbothioic acid S-fluoromethyl ester) or 6α,9α-Difluoro-11β-hydroxy-16-methyl-17-[(4-methyl-1.3-thiazole-5-carbonyl)oxy]-3-oxo-androsta-1,4-diene-17β-carbothioic acid S-fluoromethyl ester; anti-tussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; PDE4 inhibitors e.g. cilomilast or roflumilast; leukotriene antagonists eg montelukast, pranlukast and zafirlukast; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); iNOS inhibitors; a₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino]propanoic acid (e.g. as free acid or potassium salt)], diuretics, e.g., amiloride; anticholinergics, e.g., ipratropium (e.g. as bromide), tiotropium, atropine or oxitropium; ganglionic stimulants, e.g., nicotine; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Further suitable pharmaceutically acceptable agents include compounds known in the art as long acting β₂₋ adrenoreceptor agonists, particularly those generically and specifically described in patent applications WO 02/066422, WO 02/070490, WO 02/076933, PCT/GB02/004140 and PCT/GB03/002301 (all Glaxo Group Limited). Particularly preferred long acting β₂₋ adrenoreceptor agonists include 3-(4-{[6-({(2R)-2-hydroxy-2-[4-hydroxy-3-(hydroxymethyl)-phenyl]ethyl}amino) hexyl]oxy}butyl)benzenesulfonamide and 3-(3-{[7-({(2R)-2-hydroxy-2-[4-hydroxy-3-hydroxymethyl)phenyl]ethyl}-amino)heptyl]oxy}propyl) benzenesulfonamide.

Where used herein the term “pharmaceutically active agent” can also be taken to include a combination containing two or more pharmaceutically active agents of the type described above. Preferred formulations containing combinations of active ingredients contain salbutamol (e.g., as the free base or the sulphate salt) salmeterol (e.g., as the xinafoate salt), formoterol (e.g. as the fumarate salt) or a long acting β₂₋ adrenoreceptor agonists in combination with an anti-inflammatory steroid such as a beclomethasone ester (e.g., the dipropionate), a fluticasone ester (e.g., as the propionate or 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester), or budesonide.

A particularly preferred combination of active agents is fluticasone propionate and salmeterol, or a pharmaceutically acceptable salt thereof (particularly the xinafoate salt). Such a combination is described in patent EP0416951B1 (Glaxo Group Limited).

Further combinations of particular interest are budesonide and formoterol (e.g. as the fumarate salt) and also salmeterol, or a pharmaceutically acceptable salt thereof (particularly the xinafoate salt) and an anti-cholinergic such as ipratropium (e.g. as the bromide).

The quantity of active agent in the composition produced in accordance with this invention will vary significantly depending, inter alia, upon the particular active agent under consideration, the age and weight of the patient and the severity of the condition. Such considerations are familiar to the person skilled in the art. The active agent can be present in a concentration of 0.01-99%. Typically however, the active agent will be present in a concentration of 0.05 to 50%, more typically 0.1-15% of the total weight of the composition.

The excipient may be composed of particles of any pharmacologically inert material or combination of materials which is/are suitable for inhalation.

Preferred excipients include mono-saccharides, such as mannitol, arabinose, xylitol and dextrose and monohydrates thereof, disaccharides, such as lactose, maltose and sucrose, and polysaccharides such as starches, dextrins or dextrans. More preferred excipients comprise particulate crystalline sugars such as glucose, fructose, mannitol, sucrose and lactose. Especially preferred excipients are anhydrous lactose and lactose monohydrate.

Generally, the particle size of the excipient particles will be much greater than that of the inhaled active agent and as a result, do not penetrate into the respiratory tract. Thus, excipient particles for inhalable compositions may typically have particle sizes greater than 20 μm, more preferably in the range 20-150 μm.

If desired, the inhalable compositions may also contain two or more excipient particle size ranges. For example, in order to control the proportion of inhaled medicament, while retaining a good accuracy for metering, it is often desirable to use one component of the excipient that has a particle size of less than 15 μm (the fine excipient component) and another component of the excipient that has a particle size of greater than 20 μm but lower than 150 μm, preferably lower than 80 μm (the coarse excipient component).

The excipient or excipients may be commercially available in the desired particle size range or may be separated by air classification, sieving or any other method of size classification known in the art.

Preferably the weight ratio of the fine and coarser excipients components will range from 1:99 to 50:50.

Fine and coarse excipient components may consist of chemically identical or chemically different substances. The excipient mixtures may, for example, contain one chemical substance as the fine excipient and a different substance as the coarser excipient. However, the fine and coarser excipients in question may themselves constitute mixtures of different substances. Preferably the fine and coarser excipients will both be lactose.

The proportion of excipient material to be used in the inhalable compositions of this invention may vary depending upon the particular active agent, the powder inhaler for administration etc. The proportion may, for example, be about 75% to 99.5% by weight of the composition as a whole.

It will be appreciated that such inhalable compositions may also contain minor amounts of other additives e.g. taste masking agents or sweetners. It will be further appreciated that the inhalable compositions of this invention may also include yet further additives which improve stability performance, for example, magnesium stearate. Where such additives are present, they will generally not exceed 10% by weight of the total weight of the composition.

The dry powder pharmaceutical compositions in accordance with this invention can be prepared using standard methods. The pharmaceutically active agent, excipient and derivatised carbohydrate can be intimately mixed using any suitable blending apparatus, such as high shear blenders. The particular components of the formulation can be admixed in any order. Pre-mixing of particular components may be found to be advantageous in certain circumstances. The progress of the blending process can be monitored by carrying out content uniformity determinations. For example, the blending apparatus may be stopped, materials removed using a sample thief and then analysed for homogeneity by High Performance Liquid Chromatography (HPLC).

To determine the improved stability associated with compositions prepared according to this invention, the blends thus formed can be placed on accelerated stability screen (e.g. 40° C./75% relative humidity) and the fine particle fraction reduction (i.e. comparison of pre and post stability FPF data) measured as an analytical parameter using a Cascade Impactor (Cl) or Twin Stage Impinger (TSI). Such procedures are familiar to those skilled in the art.

According to the invention, the inhalable compositions can be delivered by any suitable inhalation device that is adapted to administer a controlled amount of such a pharmaceutical composition to a patient. Suitable inhalation devices may rely upon the aerosolisation energy of the patient's own breath to expel and disperse the dry powder dose. Alternatively, this energy may be provided by an energy source independent of the patient's inhalation effort, such as by impellers, patient/device created pressurised gas sources or physically (e.g. compressed gas) or chemically stored energy sources. Suitable inhalation devices can also be of the reservoir type i.e. where the dose is withdrawn from a storage vessel using a suitably designed dosing device or alternatively, inhalation devices that release drug from pre-metered units e.g. blisters, cartridges or capsules.

Packaging of the composition may be suitable for unit dose or multi-dose delivery. In the case of multi-dose delivery, the composition can be pre-metered (e.g. Diskhaler® as described in U.S. Pat. No. 4,811,731 and U.S. Pat. No. 5,035,237) or metered in use (e.g. Turbuhaler® as described in U.S. Pat. No. 4,668,218). An example of a unit-dose device is Rotahaler® (as described in U.S. Pat. No. 4,353,365).

A particularly preferred inhalation device for dry powder pharmaceutical compositions of this invention is the Diskus® inhaler (described in U.S. Pat. Nos. 5,590,645 and 5,860,149) which may be charged with blister (medicament) packs as described in U.S. Pat. No. 5,873,360. The drawings of said United States patents are specifically incorporated by reference.

The present invention therefore also provides for a medicament pack for use in an inhalation device which comprises an elongate strip formed from a base sheet having a plurality of recesses spaced along its length and a lid sheet hermetically but peelably sealed thereto to define a plurality of containers, each container having therein an inhalable composition according to the present invention.

Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

As a yet further aspect of the present invention we also provide an inhalation device for use with a medicament pack which comprises an inhalable composition according to the present invention, said device comprising:
(i) an opening station for receiving a container of a medicament pack being used with said inhalation device;
(ii) means positioned to engage peelable sheets of a container which has been received in said opening station for peeling apart the peelable sheets, to open such a container;
(iii) an outlet, positioned to be in communication with an opened container, through which a user can inhale medicament in powder form from such an opened container; and
(iv) indexing means for indexing in communication with said outlet containers of a medicament pack in use with said inhalation device.

As an alternative aspect of the present invention we also provide a medicament pack comprising a circular carrier disc which has a plurality of pre-filled, hermetically sealed containers formed integrally therewith and arranged in a circle, each container containing an inhalable composition according to the present invention, each container being puncturable to form a hole on each side thereof to allow in use, air to flow through the container to entrain the powder contained therein.

As a further aspect of the present invention there is also provided an inhalation device by which compositions of the present invention may be administered to a patient which comprises a housing, a tray mounted and capable of moving within said housing (via a plunger) adapted to receive a circular carrier disc medicament pack, an air inlet (through which air can enter said device) and an air outlet (through which a patient may inhale and receive said composition.

As an alternative aspect of the present invention we also provide a medicament pack comprising a piercable capsule which contains an inhalable composition according to the present invention.

As a further aspect of the present invention there is also provided an inhalation device by which compositions of the present invention may be administered to a patient which comprises a body shell which has a nozzle at a forward end and which is open at the rear end, a sleeve fitted on the outside of the body shell and rotatable with respect to it, a means for retaining a piercable capsule extending through the rear wall of the sleeve into the body shell, means for piercing said capsule when sleeve is rotated and a guard to ensure that the inhalable composition and not the pierced capsule, passes through the nozzle.

As a further aspect of the present invention there is also provided an inhalation device by which inhalable compositions of the present invention may be administered to a patient which comprises a nozzle, an air conduit connected to said nozzle for allowing a passage of air to be inhaled, a dosing unit comprising a storage chamber for the inhalable composition (which may also comprise a dosage indicating means) and a displaceable element for dispensing said formulation from the storage chamber into the air conduit, a manoeuvering unit for displacing said element in relation to the storage chamber and optional deflector devices to provide accelerated airflow.

In a further or alternative aspect the present invention also provides for a method of treatment or prophylaxis of respiratory disorders which comprises administering to a patient in need thereof of a dry powder pharmaceutical composition according to the present invention.

According to another aspect the present invention provides for the use of a dry powder pharmaceutical composition according to the present invention in the manufacture of a medicament for the treatment of respiratory disorders.

Suitable examples of respiratory disorders include, but are not limited to, asthma, bronchitis, chronic obstructive pulmonary disease (COPD), emphysema and rhinitis.

Preferably the respiratory disorder is asthma.

Where used herein, unless otherwise stated, the terms “dry powder pharmaceutical composition for inhalation therapy” and “inhalable composition” are to be treated as synonymous.

All publications, including but not limited to patents and patent applications, cited in this specification are herein incorporated by reference as if each individual publication were specifically and individually indicated to be incorporated by reference herein as though fully set forth.

Throughout the specification and claims which follow, unless the context requires otherwise, the word “comprise”, and variations thereof such as “comprises” and ‘comprising’, will be understood to imply the inclusion of a stated integer or step or group of integers but not to the exclusion of any other integer or step or groups of integers.

The invention will now be described in detail by way of reference only to the following non-limiting examples.

### EXAMPLE 1

Dry Powder Compositions Comprising Derivatised Carbohydrates and a 50 μg: 50 μg Combination of Salmeterol Xinafoate and Fluticasone Propionate

All derivatised carbohydrates (Aldrich, Dorset, UK) were micronised (GEM-T, Glen Creston) under nitrogen with an inlet pressure of 3.5 bar and a grinding pressure of 2.0 bar.

The blends A-E, as tabulated below, were prepared by the following procedure. All material utilised in these blends was sieved using a 500 μm aperture screen to remove large agglomerates.

Blend A, the control, is formed by mixing of lactose and actives in a 2.5L QMM (high shear) bowl for approximately 10 minutes (blend uniformity less than 4% RSD for either active material (ten samples each approx. 25 mg)).

For blends B-E, approximately half of the derivatised carbohydrates were pre-mixed with the actives and the other half pre-mixed with the lactose, both in high shear blenders. The two pre-mixes were then combined and mixing continued in a QMM blender for approximately 10 minutes. The blend uniformity data were found to be in the range 1-3% RSD for both active materials.

| Blend | Contents of blend | Amount (g) | Amount (%) |
|---|---|---|---|
| A | Salmeterol Xinafoate | 2.91 | 0.58 |
| | D(0.5) 1.6 μm* | | |
| | Fluticasone Propionate | 2.00 | 0.40 |
| | D(0.5) 2.0 μm* | | |
| | Lactose monohydrate | 495.09 | 99.02 |
| | 11.8% fines, D(0.5) 60 μm* | | |
| B | Salmeterol Xinafoate | 2.91 | 0.58 |
| | D(0.5) 1.6 μm* | | |
| | Fluticasone Propionate | 2.00 | 0.40 |
| | D(0.5) 2.0 μm* | | |
| | α-0-Sucrose Octaacetate | 35.00 | 7.00 |
| | D(0.5) 10 μm** | | |
| | Lactose monohydrate | 460.09 | 91.94 |
| | 6.5% fines, D(0.5) 84 μm* | | |
| C | Salmeterol Xinafoate | 2.91 | 0.58 |
| | D(0.5) 1.6 μm* | | |
| | Fluticasone Propionate | 2.00 | 0.40 |
| | D(0.5) 2.0 μm* | | |
| | α-D-Cellobiose Octaacetate | 35.00 | 7.00 |
| | D(0.5) 1.7 μm** | | |
| | Lactose monohydrate | 460.09 | 91.94 |
| | 6.5% fines, D(0.5) 84 μm* | | |
| D | Salmeterol Xinafoate | 2.91 | 0.58 |
| | D(0.5) 1.6 μm* | | |
| | Fluticasone Propionate | 2.00 | 0.40 |
| | D(0.5) 2.0 μm* | | |
| | D-Glucose Pentaacetate | 35.00 | 7.00 |
| | D(0.5) 4.5 μm** | | |
| | Lactose monohydrate | 460.09 | 91.94 |
| | 6.5% fines, D(0.5) 84 μm* | | |
| E | Salmeterol Xinafoate | 2.91 | 0.58 |
| | D(0.5) 1.6 μm | | |
| | Fluticasone Propionate | 2.00 | 0.40 |
| | D(0.5) 2.0 μm | | |
| | α-D-Lactose Octaacetate | 35.00 | 7.00 |
| | D(0.5) 18 μm** | | |
| | Lactose monohydrate | 460.09 | 91.94 |
| | 6.5% fines, D(0.5) 84 μm* | | |

*Laser diffraction using Malvern Mastersizer, sample dispersed in lecithin/Isooctane (Fines = material < 15 μm)
**Laser diffraction using Sympatec, Vibri sample introduction at 1 bar pressure

The blends thus formed were then added to blister packs, of the type described in patent U.S. Pat. No. 5,873,360, using filling methods according to procedures outlined in WO 00/71419 (Glaxo Group Limited). Each blister contained approximately 12 mg of the blend.

The seal integrity of the blister pack was deliberately compromised by puncturing each blister. The blister pack was then loaded into a Diskus® device.

The loaded Diskus® devices containing blends A-E were placed on accelerated stability at 40° C./75% relative humidity for period of 72 hours. Twin stage impinger analysis (in triplicate) was performed (at 60 l/min) by the method detailed in the British Pharmacopoeia (Method A) with the exception that a USP throat was substituted for the glass one and was sealed to the stage 1 jet tube using a rubber gasket. The devices were tested pre and post storage by discharging the contents of 14 blisters into the Twin Stage Impinger apparatus. The results obtained are tabulated below.

| | Pre-Storage (μg/dose) | | Post-Storage (μg/dose) | |
|---|---|---|---|---|
| | Salmeterol | Fluticasone | Salmeterol | Fluticasone |
| | base | Propionate | Base | Propionate |
| | (stage 2/ | (stage 2/ | (stage 2/ | (stage 2/ |
| Blend | emitted dose) | emitted dose) | emitted dose) | emitted dose) |
| A | 9.69/42.1 | 11.7/40.9 | 5.42/39.2 | 6.60/39.6 |
| B | 2.96/35.4 | 3.91/35.2 | 2.30/33.3 | 2.83/32.8 |
| C | 6.07/41.8 | 4.79/42.3 | 6.10/39.8 | 5.26/40.1 |
| D | 8.12/38.1 | 9.02/36.9 | 6.74/37.5 | 7.66/36.4 |
| E | 5.53/44.0 | 6.73/40. | 3.87/48.2 | 4.53/43.8 |
| | Mean Stage 2 | | Mean Stage 2 | |
| | Pre-Storage (%) | | Post-Storage (%) | |
| | Salmeterol | Fluticasone | Salmeterol | Fluticasone |
| Blend | base | Propionate | Base | Propionate |
| A | 23.0 | 28.7 | 13.8 | 16.5 |
| B | 8.35 | 11.1 | 6.91 | 8.6 |
| C | 14.5 | 11.2 | 15.3 | 13.1 |
| D | 21.3 | 24.4 | 18.0 | 21.0 |
| E | 12.6 | 16.9 | 7.98 | 10.3 |

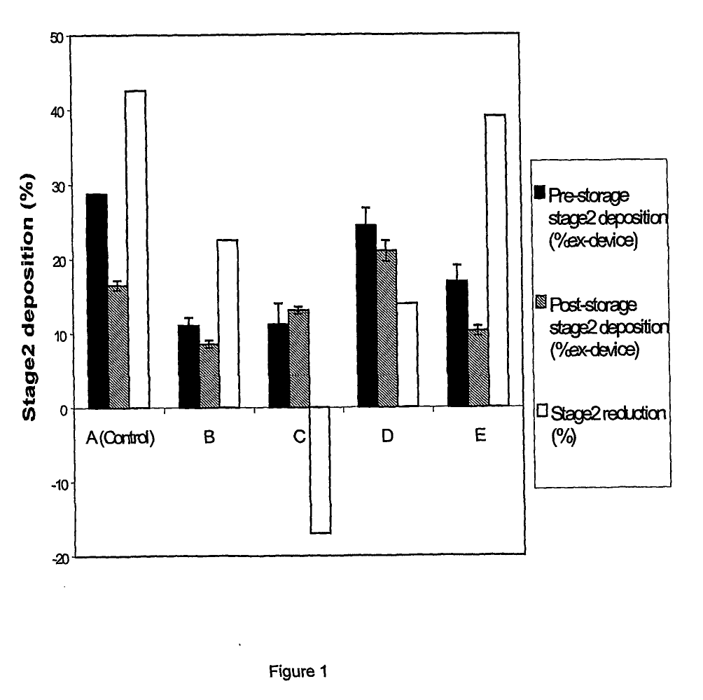

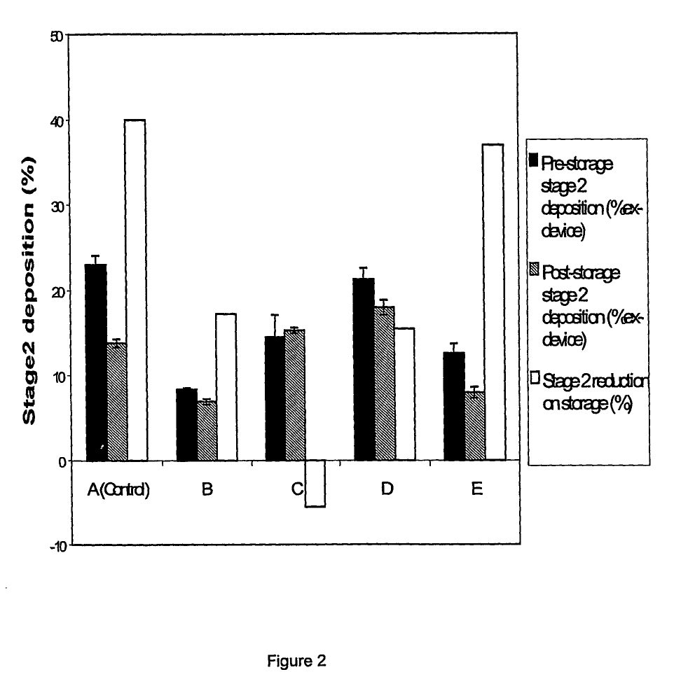

These data are represented graphically in FIGS. 1 and 2.

FIG. 1 shows the effect of derivatised carbohydrates on the twin impinger performance of the Fluticasone propionate component of Salmeterol Xinafoate/Fluticasone Propionate 50 μg/50 μg blends (+/−standard deviation).

FIG. 2 shows the effect of derivatised carbohydrates on the twin impinger performance of the Salmeterol Xinafoate component of Salmeterol Xinafoate/Fluticasone Propionate 50 μg/50 μg blends (+/−standard deviation).

### EXAMPLE 2

Dry Powder Composition Comprising Derivatised Carbohydrates and 10 μg (2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol

The pharmaceutically active agent (2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (hereafter compound A) was prepared according to procedures outlined for Example 11 of patent application WO 98/28319 (Glaxo Group Limited). The derivitised carbohydrate, trehalose diisobutyrate hexaacetate, was prepared according to procedures outlined in patent application WO 99/33853 (Quadrant Holdings). All materials were micronised.

Blend F (as control) and blends G and H were prepared using similar procedures to those detailed in Example 1.

| Blend | Contents of blend | Amount (g) | Amount % |
|---|---|---|---|
| F | Compound A D(0.5) 1.2 μm* | 0.31 | 0.105 |
| | Micronised lactose D(0.5) 6 μm** | 21.0 | 7.00 |
| | Lactose monohydrate | 278.69 | 92.9 |
| | 6.5% fines, D(0.5) 84 μm* | | |
| G | Compound A D(0.5) 2 μm* | 0.31 | 0.105 |
| | Trehalose diisobutyrate hexaacetate | 21.0 | 7.00 |
| | D(0.5) 2.5 μm** | | |
| | Lactose monohydrate | 278.7 | 92.9 |
| | 6.5% fines, D(0.5) 84 μm* | | |
| H | Compound A D(0.5) 2 μm* | 0.31 | 0.105 |
| | α-D-Cellobiose Octaacetate | 21.0 | 7.00 |
| | D(0.5) 1.7 μm** | | |
| | Lactose monohydrate | 278.7 | 92.9 |
| | 6.5% fines, D(0.5) 84 μm* | | |

*Laser diffraction using Malvern mastersizer, sample dispersed in lecithin/isooctane (Fines = material < 15 μm)
**Laser diffraction using Sympatec, Vibri sample introduction at 1 bar pressure

The blends F, G and H were tested in a similar manner to that described in Example 1 with the exception that the compromised blister packs for blends F and G were stored at 33° C./80% RH for 72 hours prior to analysis using the TSI.

| | Pre-Storage (μg/dose) | Post-Storage (μg/dose) |
|---|---|---|
| | Compound A base | Compound A base |
| Blend | (stage 2/emitted dose) | (stage 2/emitted dose) |
| F | 3.47/8.36 | 1.09/7.52 |
| G | 2.01/6.48 | 1.87/6.35 |
| H | 2.40/8.65 | 2.66/8.83 |
| | Mean Stage 2 | Mean Stage 2 |
| | Pre-Storage (%) | Post-Storage (%) |
| Blend | Compound A | Compound A |
| F | 41.5 | 14.4 |
| G | 31.0 | 29.4 |
| H | 27.7 | 30.2 |

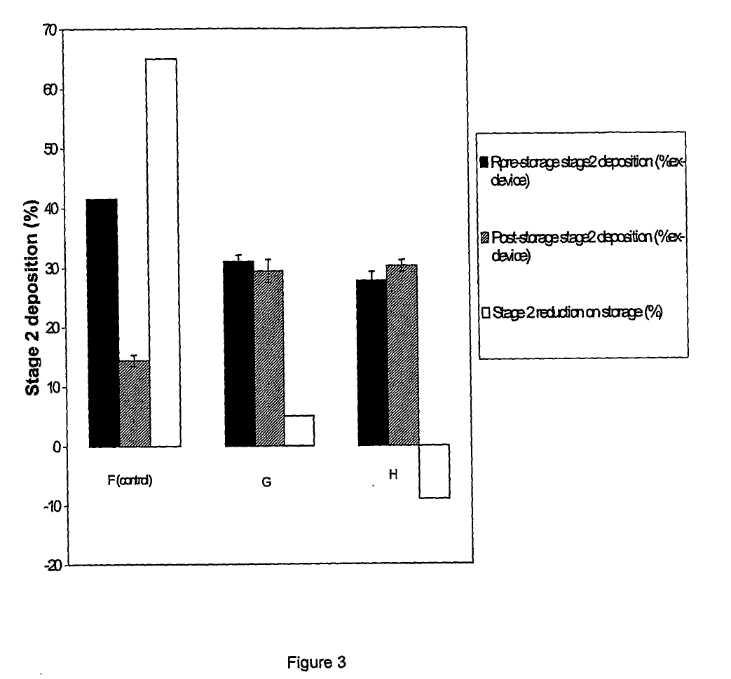

FIG. 3 shows the effect of derivatised carbohydrates on the twin impinger performance of compound A 10 μg/blister (+/−standard deviation).

### EXAMPLE 3

Dry Powder Compositions Comprising a Derivatised Carbohydrate and a 50 μg: 160 μg Combination of Salmeterol Xinafoate and Ipratropium Bromide

Blend I (as control) and blend J were prepared using similar procedures to those detailed in Example 1.

| Blend | Contents of blend | Amount (g) | Amount % |
|---|---|---|---|
| I | Salmeterol Xinafoate | 6.96 | 0.58 |
| | D(0.5) 1.6 μm* | | |
| | Ipratropium Bromide | 16.03 | 1.34 |
| | D(0.5) 1.74 μm* | | |
| | Lactose monohydrate | 1177.01 | 98.08 |
| | 10% fines, D(0.5) 68.97 μm* | | |
| J | Salmeterol Xinafoate | 6.96 | 0.58 |
| | D(0.5) 1.6 μm | | |
| | Ipratropium Bromide | 16.03 | 1.34 |
| | D(0.5) 2.0 μm | | |
| | α-D-Cellobiose Octaacetate | 84.00 | 7.00 |
| | D(0.5) 1.7 μm** | | |
| | Lactose monohydrate | 1093.01 | 91.08 |
| | 10% fines, D(0.5) 68.97 μm* | | |

*Laser diffraction using Malvern mastersizer, sample dispersed in lecithin/isooctane (Fines = material < 15 μm)

The blends I and J were tested in a similar manner to that described in Example 1 with the exception that the compromised blister packs were stored at 40° C./75% RH for 48 hours prior to analysis using the TSI.

| | Pre-Storage (μg/dose) | | Post-Storage (μg/dose) | |
|---|---|---|---|---|
| | Salmeterol | Ipratropium | Salmeterol | Ipratropium |
| | base | bromide | Base | bromide |
| | (stage 2/ | (stage 2/ | (stage 2/ | (stage 2/ |
| Blend | emitted dose) | emitted dose) | emitted dose) | emitted dose) |
| I | 7.9/42.0 | 39.3/133.3 | 2.2/27.2 | 11.6/86.4 |
| J | 16.4/40.6 | 62.4/134.8 | 16.0/38.8 | 58.7/124.6 |
| | Mean Stage 2 | | Mean Stage 2 | |
| | Pre-Storage (%) | | Post-Storage (%) | |
| | Salmeterol | Ipratropium | Salmeterol | Ipratropium |
| Blend | base | Bromide | Base | Bromide |
| I | 18.8 | 29.4 | 8.0 | 13.3 |
| J | 40.3 | 46.3 | 41.4 | 47.1 |

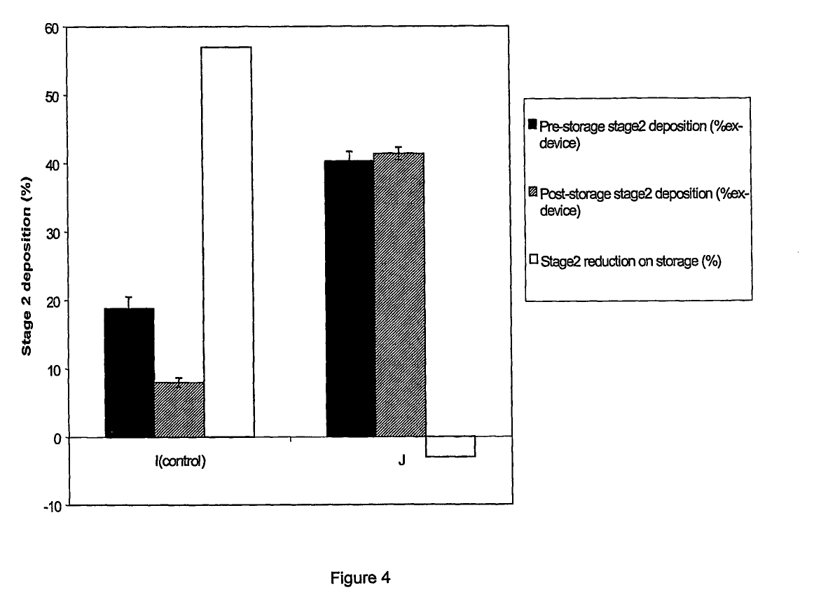

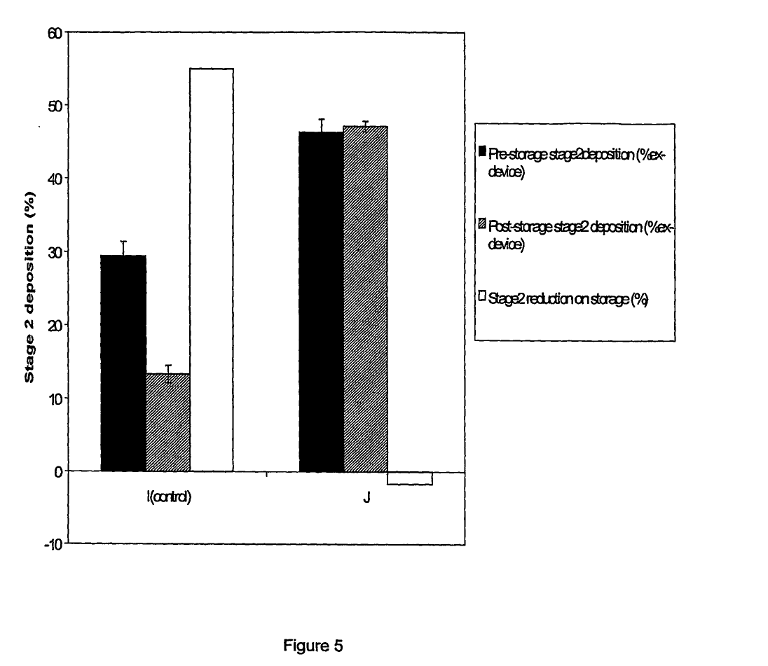

These data are represented graphically in FIGS. 4 and 5.

FIG. 4 shows the effect of derivatised carbohydrate on the twin impinger performance of the Salmeterol Xinafoate component of Salmeterol Xinafoate/Ipratropium Bromide 50 μg/160 μg blends (+/−standard deviation).

FIG. 5 shows the effect of derivatised carbohydrate on the twin impinger performance of the Ipratropium Bromide component of Salmeterol Xinafoate/Ipratropium Bromide 50 μg/160 μg blends (+/−standard deviation).

Data shown in Examples 1, 2 and 3 demonstrate that dry powder pharmaceutical compositions incorporating derivatised carbohydrates (particularly cellobiose octaacetate) can significantly reduce the deterioration in fine particle fraction following exposure to high temperature and humidity. It is believed therefore, that such compositions, when incorporated in dry powder inhaler products, would demonstrate considerably enhanced stability and hence an increased shelf-life.

Without wishing to be bound by this theory, we believe that conventional dry powder blends (e.g. those containing an active agent and excipient such as lactose), when subject to environmental humidity, result in a liquid film forming on the fine lactose particles (<15 μm) which allows dissolution of the lactose. When the humidity decreases, the lactose solution evaporates allowing the formation of permanent crystal bridges between the active agent and fine lactose particles. The resultant active agent/lactose agglomerates are not readily aerosolised and cause a reduction in the fine particle fraction. The addition of derivatised carbohydrate particles dispersed in the blend with active agent and the lactose particles may therefore prevent the formation of the crystal bridges between the fine lactose and active agent particles, hence reducing agglomeration and the consequent decline in fine particle fraction.

1. A method of improving the stability performance of dry powder pharmaceutical compositions for inhalation therapy comprising inclusion of particulate derivatised carbohydrates in said dry powder pharmaceutical compositions.

2. A method of eliminating or reducing the detrimental effect on fine particle dose resulting from storage of a dry powder pharmaceutical composition for inhalation therapy comprising inclusion of particulate derivatised carbohydrates in said dry powder pharmaceutical compositions.

3. A dry powder pharmaceutical composition for inhalation therapy comprising a pharmaceutically active agent, an excipient and a derivatised carbohydrate in particulate form.

4. A dry powder pharmaceutical composition according to claim 3 in which the derivatised carbohydrate is a mono or di-saccharide in which at least one hydroxyl group of the carbohydrate group is substituted with a hydrophobic moiety via either ester or ethers linkages.

5. A dry powder pharmaceutical composition according to claim 3 in which the derivatised carbohydrate is a carbohydrate selected from fructose, glucose, mannitol, maltose, mannitol, trehalose, cellobiose, lactose and sucrose in which at least one hydroxyl group of said carbohydrate is substituted by a straight or branched hydrocarbon chain comprising up to 20 carbon atoms.

6. A dry powder pharmaceutical composition according to claim 3 in which the derivatised carbohydrate is selected from the group consisting of cellobiose octaacetate, sucrose octaacetate, lactose octaacetate, glucose pentaacetate, mannitol hexaacetate and trehalose octaacetate.

7. A dry powder pharmaceutical composition according to claim 3 in which the derivatised carbohydrate is α-D cellobiose octaacetate.

8. A dry powder pharmaceutical composition according to claim 3 in which the derivatised carbohydrate is present at a concentration of less than 10% of the total composition.

9. A dry powder pharmaceutical composition according to claim 3 in which the derivatised carbohydrate has an aerodynamic size in the range 1-20 μm.

10. A dry powder pharmaceutical composition according to claim 3 in which one component of the excipient that has a particle size of less than 15 μm (the fine excipient component) and another component of the excipient that has a particle size of greater than 20 μm but lower than 150 μm (the coarse excipient component).

11. A dry powder pharmaceutical composition according to claim 10 in which the fine and coarse excipient components are both lactose.

12. A dry powder pharmaceutical composition according to claim 3 in which the pharmaceutically active agent is 6α,9α-Difluoro-17α-[(2-furanylcarbonyl)oxy]-11β-hydroxy-16α-methyl-3-oxo-androsta-1,4-diene-17-carbothioic acid S-fluoromethyl ester.

13. A dry powder pharmaceutical composition according to claim 3 in which the pharmaceutically active agent is 6α,9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl.

14. A dry powder pharmaceutical composition according to claim 3 in which the pharmaceutically active agent is 3-(4-{[6-({(2R)-2-hydroxy-2-[4-hydroxy-3-(hydroxymethyl)-phenyl]ethyl}amino)hexyl]oxy}butyl)benzene sulfonamide.

15. A dry powder pharmaceutical composition according to claim 3 in which the pharmaceutically active agent is 3-(3-{[7-({(2R)-2-hydroxy-2-[4-hydroxy-3-hydroxymethyl)phenyl]ethyl}-amino)heptyl]oxy}propyl) benzenesulfonamide.

16. A method of treatment or prophylaxis of respiratory disorders which comprise administering to a patient in need thereof a dry powder pharmaceutical composition according to claim 3.

17. A method of manufacture comprising inclusion of a dry powder pharmaceutical composition according to claim 3 in the manufacture of a medicament for the treatment of respiratory disorders.

18. An inhalation device containing therein a dry powder pharmaceutical composition according to claim 3.

19. An inhalation device according to claim 18 in which the dry powder pharmaceutical composition is released from a pre-metered unit medicament pack.

20. A medicament pack for use in an inhalation device which comprises an elongate strip formed from a base sheet having a plurality of recesses spaced along its length and a lid sheet hermetically but peelably sealed thereto to define a plurality of containers, each container having therein an inhalable composition according to claim 3.

21. A medicament pack according to claim 20 wherein the strip is sufficiently flexible to be wound into a roll.

22. A medicament pack according to claim 20 wherein the lid sheet and base sheet have leading end portions which are not sealed to one another.

23. A medicament pack according to claim 22 wherein at least one of the said leading end portions is constructed to be attached to a winding means.

24. A medicament pack according to claim 20 wherein the hermetic seal between the base and lid sheets extends over their whole width.

25. A medicament pack according to claim 20 wherein the lid sheet may be peeled from the base sheet in a longitudinal direction from a first end of the said base sheet.

26. An inhalation device for use with a medicament pack according to claim 20, said device comprising:
(i) an opening station for receiving a container of a medicament pack being used with said inhalation device;
(ii) means positioned to engage peelable sheets of a container which has been received in said opening station for peeling apart the peelable sheets, to open such a container;
(iii) an outlet, positioned to be in communication with an opened container, through which a user can inhale medicament in powder form from such an opened container; and
(iv) indexing means for indexing in communication with said outlet containers of a medicament pack in use with said inhalation device.

27. A medicament pack comprising a circular carrier disc which has a plurality of pre-filled, hermetically sealed containers formed integrally therewith and arranged in a circle, each container containing an inhalable composition according to claim 3 each container being puncturable to form a hole on each side thereof to allow in use, air to flow through the container to entrain the powder contained therein.

28. An inhalation device which comprises:
(a) a housing,
(b) a tray mounted and capable of moving within said housings
(c) a circular carrier disc medicament pack according to claim 27, said tray being adapted to receive said circular carrier disc medicament pack,
(d) a plunger for moving said tray,
(e) an air inlet through which air can enter said device and
(f) an air outlet through which a patient may inhale and receive said composition.

29. A medicament pack comprising a piercable capsule which contains an inhalable composition according to claim 3.

30. An inhalation device by which inhalable compositions according to claim 3 may be administered to a patient which comprises a body shell which has a nozzle at a forward end and which is open at the rear end, a sleeve fitted on the outside of the body shell and rotatable with respect to it, a means for retaining a piercable capsule according to claim 29 extending through the rear wall of the sleeve into the body shell, means for piercing said capsule when sleeve is rotated and a guard to ensure that the composition and not the pierced capsule, passes through the nozzle.

31. An inhalation device by which inhalable compositions according to claim 3 may be administered to a patient which comprises a nozzle, an air conduit connected to said nozzle for allowing a passage of air to be inhaled, a dosing unit comprising a storage chamber for the composition (which may also comprise a dosage indicating means) and a displaceable element for dispensing said composition from the storage chamber into the air conduit, a manoeuvering unit for displacing said element in relation to the storage chamber and optional deflector devices to provide accelerated airflow.
